(19) United States
(12) Patent Application Publication
(10) Pub. No.: US 2006/0121443 A1
(43) Pub. Date: Jun. 8, 2006

(54) Determination of cell viability

(75) Inventor: Francis C. Zeigler, Encinitas, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Orion Biosolutions, Inc., Vista, CA (US)

(21) Appl. No.: 11/075,901

(22) Filed: Mar. 8, 2005

Related U.S. Application Data
(60) Provisional application No. 60/633,354, filed on Dec. 3, 2004.

(51) Int. Cl. C12Q 1/00

(52) U.S. Cl. 435/4

(57) ABSTRACT

This invention provides methods and compositions for determining cell viability by use of a reagent that is detectable in viable, or living, cells even after they have been fixed. The invention may be advantageously used with a sample of cells destined for transplantation or with cells to be used in the inoculation of a culture or a fermentation batch.

### RELATED APPLICATIONS

This application claims benefit of priority from Provisional U.S. Patent Application 60/633,354, filed Dec. 3, 2004, which is hereby incorporated in its entirety as if fully set forth.

### FIELD OF THE INVENTION

This invention relates to the determination of cell viability by use of a reagent that is detectable in viable, or living, cells even after they have been fixed such that they are no longer viable. The invention thus provides methods of determining cell viability as well as identifying a cell as viable. The invention also provides compositions for use in the disclosed methods.

### BACKGROUND OF THE INVENTION

The determination of cell viability is important in a wide range of research and non-research (applied) situations. A variety of means and methods have been used to determine cell viability. Some approaches are based on the principle of viable cells being capable of excluding certain agents, such as trypan blue and ethidium monoazide. Trypan blue staining for example, is based on cell membrane integrity, which is utilized based upon a presumed correlation to cell death, which permits entry of the dye.

Other approaches are based on the principle of viable cells taking up reagents or factors that can be used to identify the cells as having been alive. Examples of this approach include the uptake of radioactive substances, such as tritium-labeled thymidine, or the uptake of a tetrazolium salt, such as the yellow tetrazolium salt MTT, which is enzymatically reduced by dehydrogenases to form insoluble purple formazan crystals by the mitochondria in metabolically active cells. The crystals are solubilized by the addition of an organic solvent, such as isopropyl alcohol or dimethyl sulfoxide, to permit color detection by spectrophotometric means. Unfortunately, the solvents used in a MTT based assay also lyse the cells to result in an overall amount of color to determine the number of viable cells.

References discussing the above methods include van de Loosdrecht, A. A., et al. J. Immunol. Methods 174: 311-320, 1994; Ohno, M., and T. Abe. J. Immunol. Methods 145:199-203, 1991; Ferrari, M., et al. J. Immunol. Methods 131: 165-172, 1990; Alley, M. C., et al. Cancer Res. 48: 589-601, 1988; Carmichael, J., et al. Cancer Res. 47:936-42, 1987; Gerlier, D., and N. Thomasset. J. Immunol. Methods 94: 57-63, 1986; and Mosmann, T. J. Immunol. Methods 65: 55-63, 1983.

U.S. Pat. No. 6,403,378 describes a method based on membrane integrity that utilizes two dyes, one of which labels all intact cells while the other labels all dead cells. The methodology permits all non-viable cells to be detected at one wavelength while all viable and non-viable cells can be detected at a different wavelength.

Citation of documents herein is not intended as an admission that any is pertinent prior art. All statements as to the date or representation as to the contents of documents is based on the information available to the applicant and does not constitute any admission as to the correctness of the dates or contents of the documents.

### BRIEF SUMMARY OF THE INVENTION

This invention provides methods and compositions for the use of a single dye to detect viable cells based upon the presence of oxidative metabolism, or the redox environment that results, in cells that are alive. The viable cells may be present within a larger population of cells containing both living and dead cells. Thus the methods may be used to directly correlate the presence of the detectable dye to the number of viable cells tested. Therefore, a direct determination of cell number and detectable dye is utilized to permit an accurate and straightforward quantification of viable cells.

In a first aspect, the invention provides a method to identify a cell as viable by contacting the cell with a pre-dye which is converted to a detectable dye in the redox environment of a viable or living cell. Preferably, the pre-dye is converted due to the presence of oxidative metabolism, such as by reaction with a reactive oxygen species (ROS) as a non-limiting example, in viable cells. Preferably, the pre-dye is converted into a dye that is detectable based on its fluorescent properties (i.e. ability to absorb light at one wavelength and then emit, or fluoresce, light at a higher wavelength).

Contacted cells are fixed such that viable cells remain stained by the converted, and now detectable, dye. Detection of the dye in fixed cells identifies them as having been viable prior to the fixing step. The fixed nature of the detectable dye and the cell advantageously provide stability in the cells such that further processing may be delayed if desired. Accordingly, the invention also provides for a method of preparing fixed cells that are detectable as having been viable by use of the steps as described herein. The ability to use fixed cells reflects a distinct benefit over situations where the dye and/or cells are not fixed and the dye may be degraded in, or lost from, the cell over time. This results in the undesirable need to evaluate the cells without significant delay.

Thus, the methods may be used to determine the number of viable cells in any cell containing sample, which may be used as a representative sampling of a larger population of cells. The invention may thus be used to determine the number, or more importantly the relative number, of viable cells in a larger population of cells. This may be advantageously applied to the determination of viable cells in a population of cells for use in transplantation as discussed herein. Such cells include cells to be introduced, or re-introduced, into a subject.

This second aspect of the invention provides a method to determine the relative number of viable cells in a population of cells by first contacting a portion, or other representative sample, of said population of cells with a pre-dye wherein said pre-dye is converted to a detectable dye in the redox environment of a viable or living cell as described above. The contacted cells are then fixed such that viable cells remain stained by the converted, and now detectable, dye. Fixed, dye-labeled, cells are then detected and used to determine the number of viable cells in the contacted portion, as a representative sample of said population of cells, based on the number of cells stained with the dye in comparison to the total number of cells in the portion. Preferably, the pre-dye is converted into a dye that is detectable based on its fluorescent properties.

The invention is advantageously used in cases of cells or tissues destined for transplantation, whether autologous or otherwise, where the testing of viability of a sample of the cells or tissue provides valuable information as to the likelihood of success in using the cells in transplantation. Non-limiting examples include the testing of insulin producing cells for transplantation into subjects with diabetes and dopaminergic cells into subjects with Parkinson's disease. The invention can also be used to evaluate the likelihood of success in using one source of donor cells or tissues versus another based upon the number or percentage of viable cells in each donor source.

Alternatively, the invention may be used to determine the number or percentage of viable cells in a “seed” culture used to inoculate a larger culture or fermentation batch. In the case of large scale fermentation of mammalian or primate or higher eukaryotic cells, the cost of the media is significantly high such that the ability to identify a “seed” culture as having larger numbers of viable cells provides the benefit of avoiding the use of poor “seed” cultures.

The methods may also be used to detect the effects of various procedures on cell viability or proliferation. Thus a treatment may be applied to a population of cells followed by a determination of the effect of the treatment on cell viability. Non-limiting examples of this additional aspect of the invention include the use of the methods disclosed herein to determine drug sensitivity, cytotoxicity of a test compound, cellular response to growth factors, and cell activation.

This aspect of the invention may be applied to test a molecule for the ability to modulate oxidative insult which alters the redox environment within a cell. Thus the invention may be used to identify a molecule as causing oxidative insult or a molecule as able to reduce or alleviate oxidative insult in the presence of a second molecule that causes oxidative insult. Embodiments of this aspect of the invention include the identification of molecules that may cause oxidative insult related to Alzheimer's disease and Parkinson's disease as non-limiting examples. With respect to the latter, the invention may be used with molecules that may be involved in the degradation of dopamine producing neuronal cells. Of course the invention can also be used to identify molecules as reducing or alleviating oxidative insult in any of these conditions.

The details of one or more embodiments of the invention are set forth in the accompanying drawings and the description below. Other features, objects, and advantages of the invention will be apparent from the drawings and detailed description, and from the claims.

### BRIEF DESCRIPTION OF THE DRAWINGS

FIG. 1 shows the results from a viability determination using human dermal fibroblasts.

### MODES OF PRACTICING THE INVENTION

This invention provides a method of identifying a cell as having been viable, said method comprising contacting a cell with an amine containing pre-dye wherein said pre-dye is converted to a detectable dye in viable cells; fixing the contacted cell such that cells that were viable remain stained by the dye; and identifying the fixed cell as having been viable by detection of the dye.

As used herein, the term “viable” refers to cells that maintain homeostasis by the use of one or more energy consuming mechanisms. Thus a “viable” cell includes those in which productive oxidative metabolism occurs to produce the necessary energy; those in which only glycolysis is used to produce energy, as well as those which maintain cellular integrity, such as the ability to exclude, or actively remove, certain molecules from the interior of the cell, by energy consuming mechanisms. Preferably, a “viable” cell is capable of undergoing mitosis, cell growth, differentiation, and/or proliferation. Of course a “viable” cell is synonymous with a “living” cell, which includes cells that are quiescent (and thus not going through the cell cycle), but nonetheless alive because energy production and consumption occurs in such cells to maintain homeostasis.

The invention also provides a method of determining the number, or the relative number, of viable cells in a population of cells, said method comprising contacting a portion of the population of cells with an amine containing pre-dye wherein said pre-dye is converted to a detectable dye in viable cells; fixing the contacted portion of cells to form fixed cells wherein cells that were viable remain stained by the dye; detecting fixed cells that fluoresce; and determining the number, or the relative number, of viable cells in said portion, as a representative sample of said population of cells, based on the number of cells stained with said fluorescent dye. In cases of determining the relative number, the number of fluorescent cells are compared to the total number of cells in the contacted portion or the number of number of cells that were not stained by the dye. This can also be used to determine the percentage of cells that are viable in the tested sample, and thus the original population of cells.

The contacting of a cell with a pre-dye may be performed at any point prior to fixation of the cell. Non-limiting examples include addition of the pre-dye to cells, tissues, or organs, after surgical removal and during disruption of the cells (or other means of isolation and/or treatment), such as by enzymatic digestion. Thus the pre-dye may be present during contacting of the cell with collagenase and/or trypsin. In some preferred embodiments of the invention, the cells are contacted with pre-dye immediately prior to, or shortly before, fixation, to identify cells as viable just before fixation.

The conversion of the pre-dye to a detectable dye in viable cells results from a chemical reaction favored by the redox environment of a living cell. This environment includes contributions to metabolic activity by mitochodria, as reflected by mitochondrial enzymes and activities. The term “redox environment” is used in relation to a system, such as that within a viable cell or tissue, that has many linked redox couples. A redox couple refers to the oxidized and reduced molecular forms that give rise to the couple. A non-limiting example is seen in the case of NAD+ and NADH, which are a redox couple that can be used to define a redox state based on the ratio of free NAD+ to free NADH. Another example of a redox couple, or pair, is glutathione disulfide (GSSG) and two molecules of glutathione (GSH). Another way to define redox state is by the half-cell reduction potential and the reducing capacity of that couple.

A redox environment is a summation of the products of the reduction potential and reducing capacity of the linked redox couples, such as that found in an organelle, a cell, a tissue, or a biological fluid, that are present. See Schafer F Q, Buettner G R. (2001) Redox state of the cell as viewed through the glutathione disulfide/glutathione couple. Free Radic Biol Med. 30:1191-1212.

This environment normally reflects the presence of oxidative metabolism in the cell, which may include the presence of a reactive oxygen species (ROS) in the cell. Non-limiting examples of ROS include singlet oxygen, hydrogen peroxide, and nitroperoxides, which are produced in viable cells. The invention also contemplates the production of ROS in living cells due to reactions other than oxidative metabolism, such as via catabolism or even anabolic activity. Oxidative metabolism refers to cellular processes which are utilized to produce energy, such as in the form of ATP or NADH or NADPH, which results in a redox environment that converts a pre-dye to a detectable dye.

The term “pre-dye” refers to a molecule which is not readily detectable and which can be converted to a “detectable dye” under appropriate conditions, such as those of the redox environment within a viable cell. Preferably, a “pre-dye” of the invention contains an amine, more preferably a primary or secondary amine, group. Alternatively, a “pre-dye” may contain a group which is converted to an amine group, such as a primary or secondary amine, within a viable cell prior to, or along with, a conversion of the “pre-dye” to a detectable dye.

A “detectable dye” is a molecule which is readily detected either directly or indirectly, preferably by virtue of its fluorescence characteristics, and which is created upon conversion of its corresponding “pre-dye” under conditions such as those within a living cell. Non-limiting examples of a “pre-dye” include non-fluorescent dihydrorhodamine 123 (CAS 109244-58-8), which is converted to fluorescent rhodamine-123 in a living cell; MTT (CAS 298-93-1, 3-(4,5-dimethylthiazol-2-yl)-2,5-diphenyltetrazolium bromide); and XTT (CAS 111072-31-2, 2,3-bis-(2-methoxy-4-nitro-5-sulfophenyl)-2H-tetrazolium-5-carboxanilide). The “detectable dye” of the invention contains an amine group, preferably a primary amine group, which can be used to fix or crosslink the dye within a cell without deleterious effect on the detectability of the dye. A preferred pre-dye of the invention is dihydrorhodamine 123.

A range of effective concentrations or amounts of the pre-dyes may be used in the practice of the invention depending upon the number, nature, and form of the cells being assayed, the nature of the pre-dye selected for use, and the conditions used in the practice of the invention. All of these factors, however, can be readily adjusted by the skilled person to determine a variety of effective concentrations or amounts, which are suitable for the detection of viable cells as described herein. Accordingly, no single range of concentrations can be stipulated for use with all cell types. As a non-limiting example offered for a better understanding of the invention, however, a range of about 100 nM to about 1, about 5 or about 10 or 100 μM, final concentration, of dihydrorhodamine 123 may be used in the practice of the invention with many cell types, including fibroblasts, epithelial cells, and neuronal cells. Thus use of dihydrorhodamine 123 at concentrations of about 100 about 200, about 300, about 400, about 500, about 600, about 700, about 800, and about 900 nM as well as about 2, about 3, about 4, about 5, about 6, about 7, about 8, about 9, about 10, about 20, about 30, about 40, about 50, about 60, about 70, about 80, about 90, and about 100 μM are contemplated for use in the practice of the invention with from about 10⁵ to about 10⁶ or 10⁷ cells. Higher concentrations are preferred for use if cells exceed 10⁷ in number. Of course the use of a higher concentration permits the use of a relatively shorter period of time in comparison to a lower concentration, which would be used for a longer period of time.

The fixing of cells containing a detectable dye as disclosed herein is preferably by use of a fixative containing an aldehyde, such as, but not limited to, formaldehyde, paraformaldehyde, glutaraldehyde, and acrolein. Non-limiting examples of such fixatives include formalin and other formaldehyde containing compositions. The use of an aldedyde is complementary to the amine, preferably a primary amine, group present in the detectable dye used in the invention because the aldehyde is capable of crosslinking the dye to other cellular components. The use of formaldehyde is preferred for the practice of the invention. Glutaraldehyde, optionally at lower concentrations or with subsequent quenching of unreacted aldehyde groups (such as with reducing agents like sodium borohydride, or by reaction with exogenous amine-containing reagents like ammonium chloride or glycine) which fluoresce, may also be used. Preferably, however, the fixative does not contain red blood cell (RBC) lysing agents.

In addition to formalin, other fixatives containing formaldehyde may be used in the practice of the invention. Such fixatives preferably contain additional agents to stabilize or extend the shelf-life of the formaldehyde. Preferably, such fixative combinations are prepared as a concentrated solution that is diluted prior to use. Non-limiting examples of such combinations as a concentrated stock include C1-C6 alcohols (from about 5 or about 10 to about 15 or 20% by weight), optionally with C1-C6 acids (about 0.1 to about 0.5% by weight), in the presence of about 37-40% (by weight) formaldehyde. The base solution for diluting fixatives may be PBS, as a non-limiting example.

After fixation, the cells may be washed in the presence of a detergent or surfactant. Washing may be used to remove unbound or excess dye from the cells, as opposed to relying upon dilution by subsequent treatment (such as dilution in the solution used for FACS). Washes with a non-ionic detergent or surfactant are preferred. Non-limiting examples of such detergents include Tween-20 (polyoxyethylene-sorbitan monolaurate), sapoinin, and Triton X-100. Using Tween-20 as a non-limiting example, the detergent concentration may be from about 0.01% to about 1%. Thus the invention includes use of detergents at about 0.01%, about 0.05%, about 0.1%, about 0.2%, about 0.3%, about 0.4%, about 0.5%, about 0.6%, about 0.7%, about 0.8%, about 0.9%, or about 1%. The detergent or surfactant may be in a base solution of PBS, optionally with sodium azide (as a preservative), as a non-limiting example.

In the practice of the invention, the cells are either in suspension or made to be in suspension, such as, but not limited to, by enzymatic digestion or dissociation, prior to fixation. This permits the advantageous ability to identify, or detect, cells labeled by the detectable dye via use of fluorescent activated cell sorting (FACS). This is a preferred embodiment of the invention because it provides the ability to detect and count the number of cells expeditiously. Alternatively, the cells in suspension may be analyzed by use of a traditional hemacytometer. In another embodiment, the cells may be analyzed directly, such as in the case of adherent cells which are treated as described herein and then used to detect the number of cells stained with a detectable dye.

In the practice of the invention with use of FACS, a sample of cells may be combined (or “spiked”) with an amount of detectable beads or particles for use as a positive control. The beads or particles can be used to determine the absolute number of counts for use in reference to the sample of cells being assayed.

Cells that may be used in the practice of the invention include any eukaryotic cell, including those isolated from a living or recently deceased subject, which may be labeled by the pre-dye/detectable dyes of the invention. Non-limiting examples include human or other mammalian or primate cells as well as a primary isolate of cells or tissues. In one embodiment of the invention, the cells are in a donor tissue or organ from one subject intended for use in a different (recipient) subject or for re-introduction into the donor. The cells may have been isolated by any appropriate method, including, but not limited to, surgical techniques to isolate cells and/or tissues. The populations of cells used in the practice of the invention include populations of any cell described herein, including populations containing insulin producing cells and dopaminergic neuronal cells.

Cells used in the present invention may also have been previously cultured in vitro or ex vivo (such as by use of tissue culture medium) prior to being use in the methods of the invention. The culture method or means may be any known or accepted in the art, so long as they are suitable to maintain or improve the viability of at least a portion of the cells being cultured. One non-limiting example is perfusion of a cell containing tissue or organ in an appropriate media to maintain or improve viability of cells in the tissue or organ. Another non-limiting example is the culturing of cells in a suitable media, such as on a plate or in suspension. While any suitable media may be used, preferred media would have reduced amounts of, or the absence of, agents which interfere with the conversion of a pre-dye to a detectable dye within a viable cell. Non-limiting examples of such an agent include antioxidants and phenol red, which is preferably omitted from culture media, such as those based on Hank's Balanced Salt Solution or Dulbecco's Modified Essential Medium (DMEM), used in the practice of the present invention. Of course culturing may be by use of an suitable device, including incubators, and chambers.

In addition to being cultured, the cells for use in the present invention may also have been otherwise treated in vitro prior to use in the methods of the invention. As non-limiting examples, the cells may have been treated ex vivo, by contact with agents which activate cell growth, agents which activate cells to differentiate one or more further steps toward a terminally differentiated phenotype, or nucleic acid containing agents which transduce the cells. Of course in some embodiments of the invention, the cells are already terminally differentiated or are believed to be terminally differentiated.

In preferred embodiments of the invention, the cells are part of a donor tissue or other cell containing material to be transplanted into a recipient subject. The cells for transplantation may have been originally obtained from the subject into which they are to be transplanted (i.e. autologous transplantation). Cells or tissues that have been cultured or otherwise treated in vitro prior to autologous transplantation may be considered to be ex vivo treated cells or tissues. Alternatively, transplantation may be of a donor tissue or other cell containing material from one subject to another.

Cells for transplantation into patients afflicted with any disease or unwanted condition may be used in the practice of the invention. Non-limiting examples include cells for use in the treatment of a neurodegenerative condition, such as Parkinson's disease, spinal cord injury, multiple sclerosis, Alzheimer's disease, Huntington's disease, and natural neuronal degeneration due to aging. Other non-limiting examples include cells for treating diabetes, leukemia, bone marrow transplantation, or hair loss as well as for use in cosmetic or reconstructive surgery.

The instant invention is particularly advantageously used in transplant situations because the number, or relative number, of viable cells in the tissue, or other cell containing material, to be transplanted may be determined based upon determination of viability of a sampling of cells from the tissue (or other cell containing material). This permits a determination of whether the cell containing material (in tissue form or otherwise) is likely to have continued viability, or further metabolic activity, growth and/or proliferation, in vivo after transplantation. Metabolic activity refers to the ability of a cell to continue utilizing energy to maintain homeostasis as well as any enhancement in the redox environment within a cell which converts pre-dye to detectable dye.

The viable cells detected by the present invention are preferably those with a therapeutically advantageous or effective phenotype or function. Non-limiting examples include insulin producing cells, dopamine producing cells, immune system cells, hemopoietic stem cells, and neural cells. Other non-limiting examples include cells for use in in vitro fermentation to produce a protein or nucleic acid product expressed by the cells or other metabolite, such as, but not limited to, steroid hormones, carbohydrates, lipids, etc., of the cells.

As stated above, cells for use in the present invention may or may not be terminally differentiated. Cells that are not terminally differentiated may be viewed as cells that are capable of further differentiation by one or more steps of differentiation. One non-limiting example of such cells is seen with progenitor cells, which are cells that have taken at least one step toward differentiation while retaining the ability to take one or more additional steps. Such cells may have retained a multipotent phenotype. Another non-limiting example is seen with stem cells, which may be multipotent or totipotent but are cells that are precursors to all progenitor cells. Progenitor cells may also be defined as “committed” and “uncommitted” cells which refer to their ability to become less differentiated by restoring the ability to take one or more steps toward differentiation toward a different terminally differentiated outcome.

Additional non-limiting examples of such cells include hematopoietic stem cells, bone marrow cells, umbilical cord blood cells, neural stem cells, neural progenitor cells, adult or embryonic or fetal stem cells, and embryonic stem cells. Non-limiting examples of neural stem or progenitor cells include nestin expressing neuroepithelial cells or radial glial cell-like neuroglial progenitor cells.

Of course the invention may also be practiced with cells derived from any stem or progenitor cell. Such cells may have been derived, or differentiated, during culture in vitro (or ex vivo) in the absence of any intentional stimulation or contact with an exogenous factor or agent. Alternatively, the cells may have been contacted in vitro or ex vivo with an exogenous factor or agent which stimulates differentiation via taking one or more steps toward terminal differentiation.

Non-limiting examples of terminally differentiated cells for use in the present invention include insulin producing cells, such as pancreatic islet cells, dopamine producing cells, hepatocytes, neurons, motor neurons, glial cells, lymphocytes (including B and T cells), leukocytes, monocytes, granulocytes (neutrophils, eosinophils, basophils), phagocytes, fibroblasts, skin cells, hair cells, epithelial cells, and oligodendrocytes.

Other cells for use with the instant invention include mesenchymal stem cells, fibroblasts, chondrocytes, keratinocytes, endothelial cells, smooth muscle cells, macrophages, pancreatic stem cells, astrocytes, 3T3 cells, 293 cells, COS cells, CHO cells, MEF (mouse embryonic fibroblasts), HUVEC (human umbilical vein endothelial cells), CaCo-2, peripheral blood mononuclear cells (PBMCs) or types thereof, pancreatic alpha cells or beta cells, or exocrine or ductal cells found in pancreatic islets.

In embodiments of the invention to detect the effects of a test compound or drug on cell viability or the intracellular redox environment, the invention provides a method comprising contacting cells with an amine containing pre-dye simultaneous with or after said cells are contacted with a test compound or drug, wherein said pre-dye is converted to a detectable dye in viable cells; fixing the contacted cells to form fixed cells wherein cells that were viable remain stained by the dye; detecting fixed cells that fluoresce; determining the number, or the relative number, of viable cells based on the number of cells stained with said fluorescent dye; and comparing said number or relative number to another sample of cells not contacted with said test compound or drug. In cases of determining the actual number of viable cells, the cells tested with and without contacting the test compound or drug should be as identical as possible. In cases of determining the relative number, the number of fluorescent cells are compared to the total number of cells in the contacted portion or the number of number of cells that were not stained by the dye.

In preferred embodiments of the invention, the cells are treated with the test compound or drug before being contacted with a pre-dye and processed as described herein. As a non-limiting example, candidate molecules that are to be tested for a potential cytotoxic effect may be placed in contact with cells, optionally over a range of concentrations or amounts in individual applications of the invention, prior to being contacted with a pre-dye and processed as disclosed. Similarly, candidate molecules that are to be tested for a potential effect on the cellular redox environment may be similarly used in the practice of the invention.

Non-limiting examples of test compounds and drugs include growth factors, cytokines, steroid hormones, cell surface binding molecules (e.g. ligands that bind a cell surface receptor), antibodies that bind the cell surface, antitumor agents, anti-proliferative agents, extracts of naturally occurring biological materials, compounds isolated from naturally occurring biological materials, and small organic molecules produced synthetically or isolated from naturally occurring sources. In other preferred embodiments of the invention the test compound or drug is one that may cause oxidative insult to cells, particularly insulin producing cells or dopamine producing neurons.

Of course the ability of a test compound or drug to counteract or alleviate the cytotoxic effect of another agent may also be tested. In such embodiments of the invention, the method comprises contacting cells with an amine containing pre-dye simultaneous with or after said cells are contacted with a test compound or drug and a cytoxic agent, wherein said pre-dye is converted to a detectable dye in viable cells; fixing the contacted cells to form fixed cells wherein cells that were viable remain stained by the dye; detecting fixed cells that fluoresce; determining the number, or the relative number, of viable cells based on the number of cells stained with said fluorescent dye; and comparing said number or relative number to another sample of cells not contacted with said test compound or drug. Of course the cytotoxic agent may be placed in contact with the cells after the contacting with the test compound or drug. This permits the identification of the test compound or drug as having a preventive, or prophylactic effect relative to the cytotoxic agent. Alternatively, the cytotoxic agent may be placed in contact with the cells simultaneously with contacting the cells with the test compound or drug. This permits the identification of the test compound or drug as having a rapid effect in counteracting or alleviating the cytotoxic effect of the agent.

As a non-limiting example, oxidative stress from radicals such as superoxide has been associated with neuronal cell death and neurodegenerative conditions such as Parkinson's disease. Mouse embryonic stem cells (ESCs) have been used to generate dopaminergic neurons deficient in DJ-1 (see Martinat et al. PLoS Biol. 2(11):e327 (2004)). These cells have enhanced sensitivity to oxidative stress and mimic the mutation found in human inherited Parkinson's disease. DJ-1 has also been linked to the aggregation of alpha-synuclein, which is also associated with Parkinson's disease. The present invention may be applied to such mouse ESCs as well as any human or other mammalian cells to identify or screen for new neuroprotectant drugs which modulate or reduce the effects of oxidative stress in the cells. Such use in human ESCs or human adult or fetal stem cells are preferred embodiments of the invention.

As an additional non-limiting example, the effect of an activation agent on cells is contemplated for use with the present invention. Non-limiting activation agents for use with immune system cells include phorbol 12-myristate 13 acetate, ionomycin, Staphylococcal enterotoxin B (SEB), and brefeldin-A.

The invention also provides for a further modification to identify or screen for an agent that causes oxidative insult or injury to a cell. Such an assay to may be helpful in identifying agents with anticancer or antitumor activity.

As in the case above, cells tested with and without contacting the test compound or drug should be as identical as possible where determining the actual number of viable cells is used. Where the relative number of cells is determined, the number of fluorescent cells are compared to the total number of cells in the contacted portion or the number of number of cells that were not stained by the dye.

The invention further provides for articles of manufacture to identify or detect viable cells. An article of manufacture according to the present invention may be a kit for the practice of the methods disclosed herein or an article containing one or more reagents needed to practice the methods. The kit can comprise the pre-dye and/or fixative, as well as optionally one or more other reagents, for use in the present invention, together with suitable packaging material. Preferably, the packaging includes a label or instructions for the use of the article or kit in a method disclosed herein.

Having now generally described the invention, the same will be more readily understood through reference to the following examples which are provided by way of illustration, and are not intended to be limiting of the present invention, unless specified.

### EXAMPLES

The following examples are offered to illustrate, but not to limit the claimed invention.

### Example 1

Materials and Methods

All materials may be obtained from any suitable source, including those available from multiple commercial entities. Many pre-dyes are available from Molecular Probes as well as other vendors.

IntraCyte-Fix™ from the IntraCyte™ Intracellular FACS Kit available from Orion Biosolutions (Vista, Calif.) may be used as the fixative in the following.

General Protocol

For cells that are not normally in suspension, prepare single-cell suspension by enzymatic digestion or dissociation, preferably sufficiently mild to minimize cell damage. As a non-limiting example, adherent cells may be incubated cells with trypsin/EDTA in a buffered salt solution at physiological pH, such as by use of trypsin/EDTA in Hank's Balanced Salt Solution (HBSS) without Ca++/Mg++ or phenol red (Sigma Cat. # H-6648). Trypsin may be present from 0.2 to 0.01% (w/v) while EDTA may be present from 0.02 to 0.001% (w/v) as non-limiting examples.

In cases of organs or tissues, collagenase, at 0.5 to 2 mg/ml in Ca++ containing balanced salt solutions at physiological pH (e.g. DMEM) may be used prior to trypsinization. As a non-limiting example, pancreatic islets may be first liberated from pancreata using a collagenase perfusion, with isolated islets being subsequently trypsinized. Sigma Cat. # T-4174 diluted 25-fold in HBSS may be used for isolated islets at 20 to 28° C. (room temperature) for 20 minutes with occasional mixing, followed by gentle trituration with 5 ml serological pipet.

Trypsin activity may be neutralized by adding an equal volume of protein-containing HBSS (0.1% BSA or 1% serum added). Suspended cells may be centrifuged at approximately 200 g for 5 to 10 minutes, optionally at reduced temperature, to concentrate them.

Of course in cases with cells already in suspension, the above enzymatic steps may be omitted such that only the concentration step, if necessary, is used.

After removal of media, the cells are resuspended. As a non-limiting example, the cells are resuspended in 37° C. pre-warmed DMEM/F12 without phenol red or added protein (Gibco Cat. #21041-025). Preferably, a sample of unstained cells, e.g. about 5×10⁴ as a non-limiting example, are removed at this point for visual inspection or use in FACS to determine the levels of background fluorescence.

All or part of the resuspended cells are contacted with a pre-dye. Alternatively, the pre-dye was present during collagenase perfusion and/or digestion with trypsin. As a non-limiting example, dihydrorhodamine 123 (Molecular probes Cat. # D23806) is added to a final concentration of 1 to 5 μM from a 5 mM stabilized stock. The cells are gently mixed. Optionally, aliquots of the stock pre-dye may be stored as single-use small aliquots at −20° C.

For immediate use, the cells are incubated for 15 to 30 minutes at 37° C. or other suitable temperature. A sample of cells, e.g. about 5×10⁴ as a non-limiting example, may be removed at this point for FACS analysis. In cases with dihydrorhodamine 123, the cells are analyzed with 488 nm excitation in FL1 channel (525 nm band pass filter). This “live” sample analysis is not required, but is helpful to control for loss of non-viable cells after fixation. The unstained sample described above is used to set background fluorescence levels in first decade of log scale in FL1.

The remaining cells are washed, optionally with DMEM/F12 followed by centrifuging/concentration as described above followed by resuspension, such as in 1 ml HBSS as a non-limiting example. The cells may then be fixed with an appropriate fixative, such as one containing formaldehyde. As a non-limiting example, 4 ml of formalin is added to 1 ml of resuspended cells followed by gentle mixing. Fixation is permitted to continue in the dark for any appropriate length of time, such as, but not limited to, at least 3 hours at about 20 to about 28° C. (room temperature), or overnight at 4 to 10° C. in refrigerator.

After fixation, the cells may be immediately used in FACS or alternatively washed, optionally in the presence of a non-ionic detergent, and concentrated again prior to FACS. For the latter, the cells may be, as a non-limiting example, washed in HBSS or PBS (phosphate buffered saline) by adding at least 3 volumes and centrifuging at 300 to 400 g for 10 minutes at 15 to 25° C.

### Example 2

Determination of Human Dermal Fibroblast Viability

Primary human dermal fibroblasts (approximately 95% viable by trypan blue dye exclusion) were treated essentially as described in the previous example with or without addition of pre-dye (dihydrorhodamine 123) and with or without treatment with IntraCyte-Fix™ from the IntraCyte™ Intracellular FACS Kit available from Orion Biosolutions (Vista, Calif.).

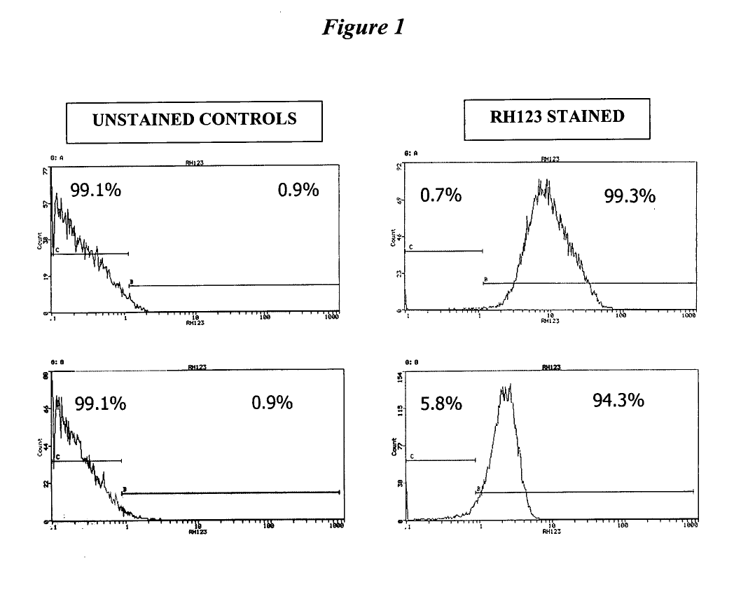

The results are shown in FIG. 1, where the horizontal axes are rhodamine 123 fluoresence and the vertical axes are cell counts. The two upper panels show the results without use of fixation while the two lower panels show the result post fixation. Prior to FACS, cells in the upper panels were washed with PBS in the absence of detergent to prevent undue leakage of dye from the cells, while cells in the lower panels were washed in the presence of about 0.1% Tween-20.

As can be seen from the two lower panels, cells that were viable stained well with rhodamine 123 (RH123) after fixation. They allowed determination of the viable cells to have been about 94% of the cell population. In contrast, the unfixed cells indicated over 99% of the cells as stained by RH123.

All references cited herein are hereby incorporated by reference in their entireties, whether previously specifically incorporated or not. As used herein, the terms “a”, “an”, and “any” are each intended to include both the singular and plural forms.

Having now fully described this invention, it will be appreciated by those skilled in the art that the same can be performed within a wide range of equivalent parameters, concentrations, and conditions without departing from the spirit and scope of the invention and without undue experimentation. While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method of identifying a cell as having been viable, said method comprising
contacting a cell with an amine containing pre-dye wherein said pre-dye is converted to a fluorescent dye in viable cells;
fixing said contacted cell wherein viable cells remain stained by said fluorescent dye;
identifying said fixed cell by use of fluorescent activated cell sorting (FACS) as having been viable by detection of fluorescence from said dye.

2. The method of claim 1 wherein said cell has been previously subjected to an isolation procedure or treatment.

3. The method of claim 2 wherein said isolation procedure is surgical isolation or enzymatic dissociation.

4. The method of claim 1 wherein said cell is a pancreatic islet cell.

5. The method of claim 1 wherein said cell is a dopaminergic neuronal cell.

6. A method of determining the relative number of viable cells in a population of cells, said method comprising
contacting a portion of said population of cells with an amine containing pre-dye wherein said pre-dye is converted to a fluorescent dye in viable cells;
fixing said contacted portion of said population of cells to form fixed cells wherein cells that were viable remain stained by said fluorescent dye;
detecting fixed cells that fluoresce by use of fluorescent activated cell sorting (FACS); and
determining the relative number of viable cells in said portion, as a representative sample of said population of cells, based on the number of cells stained with said fluorescent dye in comparison to the total number of cells in said portion.

7. The method of claim 6 wherein said pre-dye is converted to a detectable dye by a reactive oxygen species (ROS) in viable cells.

8. The method of claim 1 wherein said pre-dye is non-fluorescent dihydrorhodamine 123, which forms fluorescent rhodamine-123 in viable cells, MTT, or XTT.

9. The method of claim 1 wherein said fixing is with a fixative comprising an aldehyde.

10. The method of claim 1 wherein said fixative comprises formaldehyde or formalin.

11. The method of claim 6 wherein said population of cells is a population of cells for transplantation.

12. The method of claim 11 wherein said population of cells for transplantation are cells capable of further differentiation.

13. The method of claim 11 wherein said population of cells for transplantation are insulin producing cells, pancreatic islet cells, dopamine producing cells, hepatocytes, neurons, motor neurons, glial cells, lymphocytes, leukocytes, fibroblasts, skin cells, hair cells, epithelial cells, or oligodendrocytes.

14. The method of claim 12 wherein said population of cells are hematopoietic stem cells or bone marrow cells.

15. The method of claim 12 wherein said population of cells for transplantation are neural stem or progenitor cells.

16. The method of claim 15 wherein said neural stem or progenitor cells are nestin expressing neuroepithelial cells or radial glial cell-like neuroglial progenitor cells.

17. The method of claim 11 wherein said population of cells for transplantation are a population of cells derived from neural stem or progenitor cells.

18. The method of claim 6 wherein said population of cells has been cultured or modified ex vivo.

19. The method of claim 11 wherein said population of cells for transplantation are suitable for treatment of a neurodegenerative condition, diabetes, Parkinson's disease, spinal cord injury, multiple sclerosis, Alzheimer's disease, Huntington's disease, leukemia, hair loss, and natural neuronal degeneration due to aging.
